(19) United States
(12) Patent Application Publication
(10) Pub. No.: US 2017/0367662 A1
(43) Pub. Date: Dec. 28, 2017

(54) MULTI-LAYER ALARMING

(71) Applicant: Covidien LP, Mansfield, MA (US)

(72) Inventor: ROBERT T. BOYER, Longmont, CO (US)

(21) Appl. No.: 15/195,697

(22) Filed: Jun. 28, 2016

(51) Int. Cl. A61B 5/00; A61B 5/11; A61B 5/08; A61B 5/021; A61B 5/145; A61B 5/01; A61B 5/024

(52) U.S. Cl. CPC A61B 5/746; A61B 5/7405; A61B 5/14542; A61B 5/1118; A61B 5/01; A61B 5/0816; A61B 5/021; A61B 5/024

(57) ABSTRACT

Methods, systems, and devices for wireless patient monitoring are described. The methods, systems, and devices, provide a multi-layer alarm having multiple layers that indicate information associated with a patient being monitored by medical devices. The multi-layer alarm may be encoded with at least two layers of information, one layer indicating information related to severity of a measured parameter of the patient, and a second layer indicating other information related to the patient. The multi-layer alarm may be audibly transmitted allowing a clinician to be alerted of relevant information relating to the patient being monitored.

### BACKGROUND

The following relates generally to alerting a clinician, and more specifically to multi-layer alarming.

In a healthcare facility such as a hospital, physiological parameters of the patient (e.g., heart rate, respiratory rate, blood pressure) may be monitored by one or more medical devices. The medical devices may be battery powered and may wirelessly transmit measured patient data over a wireless network within the hospital, thereby allowing the patient to move freely through the hospital while being monitored. Clinicians may remotely monitor the patient by accessing the patient data at a central nurse station or on any web enabled device connected to the network (e.g., smartphone or tablet). Other information related to the patient (e.g., location information, patient identification information, responsible clinician) may also be recorded and stored at a central nurse station or otherwise made available to clinicians.

When multiple patients are being monitored, alarms from several medical devices may be transmitted and sounded at a central nurse station or on a mobile device carried by a clinician. Because multiple alarms from multiple patients may be sounding simultaneously, it may be difficult for a clinician to discern relevant patient information or determine clinician responsibility simply by listening to the alarms. Also, the multiple alarms at the nurse station may contribute to alarm fatigue, because each clinician can hear all of the alarms, regardless of who is the responsible clinician or whether the alarm requires immediate attention. If the sounding alarm is urgent, any delay in responding to the alarm could put a patient or multiple patients at risk.

### SUMMARY

The described features generally relate to methods and devices for alerting a clinician by sounding an alarm having multiple layers (i.e., a multi-layer alarm). Each layer of the multi-layer alarm may indicate certain information associated with a patient being monitored by one or more medical devices. For example, a first layer of the multi-layer alarm may identify a severity of a measured physiological parameter, such as heart rate, blood pressure, temperature, respiratory rate, activity level, or oxygen saturation level of the patient. A second layer of the multi-layer alarm may indicate other information such as a clinician responsible for the patient being monitored.

To alert a clinician, the multi-layer alarm may be audibly transmitted (e.g., using a speaker). Each layer of the multi-layer alarm may correspond with layer properties such as pitch, volume, and periodicity, which define each layer and different each layer from another layer of the multi-layer alarm.

Embodiments of systems and devices for multi-layer alarming are also described. In accordance with certain aspects, an apparatus for multi-layer alarming includes a processor, memory in electronic communication with the processor, and instructions stored in the memory. The instructions stored in the memory may be operable, when executed by the processor, to cause the apparatus to encode a multi-layer alarm associated with a patient, encode a first audible layer of the multi-layer alarm, the first audible layer identifying a severity of a measured parameter of the patient, encode a second audible layer of the multi-layer alarm, the second audible layer indicating information other than the identified severity of the measured parameter, and audibly transmit the multi-layer alarm.

### BRIEF DESCRIPTION OF THE DRAWINGS

FIG. 1 illustrates an example of a system for wireless patient monitoring that supports multi-layer alarming in accordance with aspects of the present disclosure.

FIG. 2 illustrates an example of a system for wireless patient monitoring that supports multi-layer alarming in accordance with aspects of the present disclosure.

FIG. 3 illustrates an example of a multi-layer alarm in accordance with aspects of the present disclosure.

FIGS. 4A-4D illustrate examples of a multi-layer alarm in accordance with aspects of the present disclosure.

FIGS. 5 through 7 show block diagrams of a device that supports multi-layer alarming in accordance with aspects of the present disclosure.

FIG. 8 illustrates a block diagram of a system including a device that supports multi-layer alarming in accordance with aspects of the present disclosure.

FIGS. 9 and 10 illustrate methods for multi-layer alarming in accordance with aspects of the present disclosure.

### DETAILED DESCRIPTION

In a healthcare facility, multiple alarms from several patients may be sounding at a central nurse station, each of which may pertain to different information of the patient. The alarms may be sound from a variety of different medical devices used to monitor the patient. In some cases, multiple medical devices may be used to monitor a single patient, each of which may sound an alarm based on certain criteria. For example, a medical device may be used to monitor the heart rate of a patient and may sound when the heart rate falls below a minimum threshold. Another medical device may be used to monitor the respiratory rate of the patient and may sound when the respiratory rate reaches a maximum threshold.

When multiple alarms are being sound from multiple medical devices monitoring a patient, the clinician(s) responsible for the patient may be unable to discern relevant information (such as whether the patient needs immediate attention) from the multiple alarms. Further, if a clinician or group of clinicians are responsible for monitoring multiple patients, it may also be difficult to discern which alarm corresponds to a particular patient when multiple alarms are being sound. However, by generating a multi-layer alarm in accordance with aspects of the present disclosure, a clinician or a group of clinicians monitoring one or more patients may be able to discern relevant information and responsibilities more quickly and accurately.

In one or more embodiments, the multi-layer may be encoded with multiple audible layers, each of which being associated with layer properties such as pitch, volume, and periodicity. Each layer of the multi-layer alarm may indicate information related to a patient being monitored. Once generated, the multi-layer alarm may be audibly transmitted in order to alert one or more clinicians of relevant information relating to the patient.

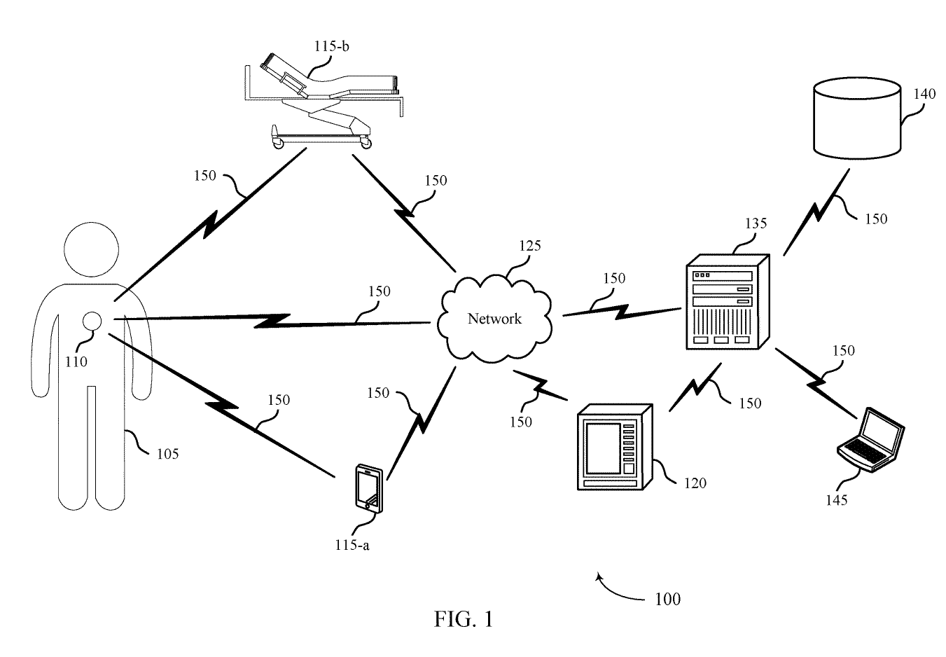

FIG. 1 illustrates an example of a wireless patient monitoring system 100 in accordance with various embodiments of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless communications links 150 to computing devices 115 or to a network 125. In some cases, the medical device 110 may be used in conjunction with another medical device 110 to generate a multi-layer alarm.

Computing device 115-a may be a wireless device such as a tablet, cellular phone, personal digital assistant (PDA), dedicated receiver, a wireless laptop computer, a mobile computer station or other similar device or a spatially distributed network of devices configured to receive signals from the medical device 110. Computing device 115-b may be an in-room patient monitoring device, a Workstation on Wheels, or a smart hospital bed which may be linked to the network 125 and medical device 110 using wireless links 150. Computing device 115-b may be configured to gather or receive data associated with the patient 105 using multiple sources and may also include a medical device 110 used to monitor parameters or collect data from the patient 105. The computing devices 115 may be in communication with a central station 135 via network 125.

The medical device 110 may also communicate directly with the central station 135 via the network 125. The central station 135 may be a server located within the hospital or in a remote location and may be in communication with a nurse station 120 using wireless links 150. The central station 135 may be in further communication with one or more remote computing devices 145, thus allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

The medical device 110 may include one or more sensors configured to collect a variety of physiological parameters as well as information related to the location and movement of the patient 105. For example, the medical device 110 may include a pulse oximetry (SpO2) sensor, a heart rate sensor, a blood pressure sensor, a pressure sensor, an electrocardiogram (ECG) sensor, a respiratory rate sensor, a glucose level sensor, a body temperature sensor, an accelerometer, a global positioning sensor, a sensor which triangulates position from multiple computing devices 115, or any other sensor configured to collect physiological, location, or motion data.

The medical device 110 may be coupled with the patient 105 in a variety of ways depending on the data being collected. For example, the medical device 110 may be directly coupled with the patient 105 (e.g., physically connected to the patient's chest, worn around the patient's wrist, or attached to the patient's finger). The sensor may be indirectly coupled with the user so that movement of the patient 105 is detected even though the sensor is not in direct contact with, or physically connected to, the patient 105 (e.g., the medical device 110 may be disposed under the patient 105). The data collected by the medical device 110 may be wirelessly conveyed to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

In accordance with various embodiments, the nurse station 120 may be used to monitor the patient 105 and may obtain data associated with the patient 105 from the medical device 110. Data may also be obtained by the nurse station 120 from the network 125 or from the server 135 or remote databases 140 using wireless links 150 or may be obtained from the medical device 110 or computing devices 115. The obtained data may then be displayed or otherwise accessible using the nurse station 120 so that nurses (or other clinicians) are able to monitor the patient 105 (and other patients).

The medical device 110 may be configured to trigger or sound an alarm based on certain criteria associated with the medical device and the patient. In some cases, the alarm is sound at the medical device 110 within the room of the patient 105. Additionally or alternatively, the medical device 110 may trigger an alarm to be sent to a remote location (e.g., nurse station 120), where the alarm is sound. The medical device 110 may monitor a physiological parameter of the patient such as heart rate, respiratory rate, blood pressure, etc., and may trigger or sound an alarm when the measure physiological parameter crosses a threshold. The medical device 110 may also sound an alarm based on a status of the medical device. For instance, if the medical device 110 experiences a technical issue such as a startup failure or has a low battery, an alarm may be triggered or sound.

In some aspects, the medical device 110, the nurse station 120, or the computing devices 115 may be configured to generate and/or audibly transmit a multi-layer alarm. For example, the nurse station 120 or the computing devices 115 may be linked to the medical device 110 and may sound a multi-layer alarm based on data received from the medical device 110. In some embodiments, the multi-layer alarm may be sound at the nurse station 120 or the computing devices 115 in conjunction with or based on the alarm from the medical device 110.

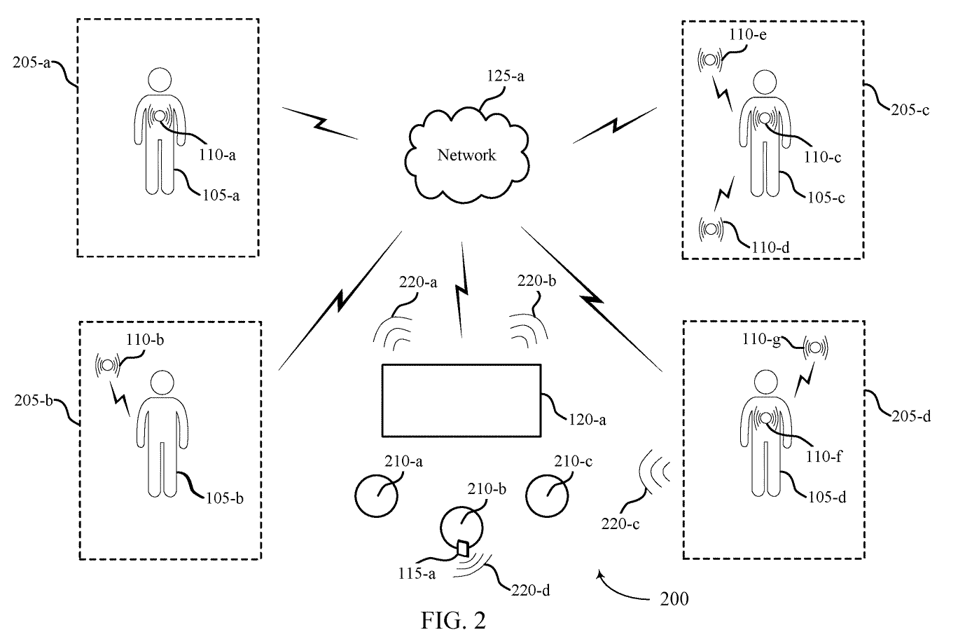

FIG. 2 illustrates an example of a wireless patient monitoring system 200 for multi-layer alarming. In some cases, the wireless patient monitoring system 200 may represent aspects of techniques performed by a computing devices 115, medical devices 110, or nurse station 120 as described with reference to FIG. 1.

As shown in FIG. 2, multiple patients 105 are located in different rooms 205 and may be monitored by one or more clinicians 210. In FIG. 2, patient 105-a is located in room 205-a, patient 105-b is located in room 205-b, patient 105-c is located in room 205-c, and patient 105-c is located in room 205-c. The rooms 205 may be located throughout a healthcare facility (e.g., each room may be on a different floor of a hospital) or may be located in different healthcare facilities remote from clinicians 210. For example, patient 105-a in room 205-a may be located at home, patients 105-b and 105-c may be located in an intensive care area of a healthcare facility, and patient 105-d may be located in room 205-d in a healthcare facility different from the other patients 105-b and 105-c, but in the same area (e.g., the same floor of a hospital) as nurse station 120-a.

Clinicians 210 may monitor patients 105 using medical devices 110, nurse station 120-a, or other devices such as computing device 115-a. Each patient 105 may be monitored by medical devices 110 connected to, or otherwise associated with, a patient 105. For example, medical device 110-a may be worn by patient 105-a to monitor heart rate of the patient 110-a. Medical device 110-b may not be directly attached to patient 105-b, but may indirectly measure movement of patient 110-b. Patient 105-c may be monitored by multiple medical devices 110-c, 110-d, and 110-e. As shown, medical device 110-c is directly attached to patient 105-c and may monitor a physiological parameter of the patient 105-c. Medical devices 110-d and 110-e may indirectly monitor other parameters of the patient 105-c. While each of medical devices 110-c, 110-d, and 110-e may measure different parameters of the patient 105-c, in some cases, medical devices 110-c, 110-d, and 110-e may be used in conjunction with one another to monitor one or more parameters associated with the patient 105-c. Patient 105-d may be monitored using medical devices 110-f and 110-g, each of which may be configured to monitor different parameters associated with the patient 105-d or may be used in conjunction with each other to monitor one or more parameters of the patient 105-d.

In some cases, medical devices 110 may be used in conjunction with other devices to monitor the patients 105. Medical devices 110 may wirelessly communicate with one another through network 125-a or may be directly connected to each other when monitoring patients 105. Medical devices 110 may also communicate with nurse station 120-a or computing device 115-a so that nurses 210 are able to monitor patients 105 from one location even though the patients 105 are in different locations. For example, medical devices 110 may transmit data to nurse station 120-a or computing devices 115-a via network 125-a. Alternatively, nurse station 120-a may request or retrieve data from one or more medical devices 110 without the medical devices 110 having to transmit the data. For example, data from a medical device 110 may be stored on the medical device 110 and may only be viewable by a responsible clinician 210. The responsible clinician 210 may then use nurse station 120-a to access or retrieve the data stored on medical device 110. The transmitted or retrieved data may then be viewable or otherwise accessible by clinicians 210 to monitor the patients 105.

The data from one or more medical devices 110 may include alarms that may be triggered when a parameter being measured by a medical device 110 crosses a threshold, for example. In some cases, the medical device 110 may sound an alarm or may transmit information to the nurse station 120-a or computing device 115-a triggering the nurse station 120-a or computing device 115-a to also sound an alarm. The thresholds for sounding an alarm may be patient specific in that each patient 105 may be associated with different thresholds for different medical devices 110 used to monitor the patient 105. For example, patient 105-a may be in room 205-a at home and may be relatively active compared to patient 105-c in intensive care. Therefore, the heart rate of patient 105-a monitored by medical device 110-a may have a relatively high threshold for sounding (or triggering) an alarm compared to the threshold for an alarm associated with patient 205-c (monitored by medical device 110-c, for example).

In one or more embodiments, nurse station 120-a may obtain data from medical devices 110 for monitoring patients 105. The nurse station 120-a may sound an alarm based on information received or obtained from medical devices 110. Further, multiple alarms may be sound from the nurse station 120-a for each patient 105 or for multiple patients 105. For example, data from medical device 110-a may cross a threshold for patient 105-a and an alarm 220-a may be sound from nurse station 120-a. In addition, data from medical devices 110-c and 110-d may also cross a threshold for patient 105-c, and an alarm 220-b (or multiple alarms associated with each medical device 110-c and 110-d) may be sound from nurse station 120-a. Medical device 110-g monitoring patient 105-d may also sound an alarm 220-c from room 205-d, which is in the same area as nurse station 120-a. Computing device 115-a may be a mobile computing device 115-a for clinician 210-b and may communicate with medical devices 110 or nurse station 120-a. In some cases, computing device 115-a may also sound an alarm 220-d based on one or more medical devices 110 or the alarms 220-a or 220-b from nurse station 120-a. In such situations, multiple alarms 220 from multiple sources (nurse station 120-a, room 205-d, computing device 115-a, etc.) may be sound at the same time which may lead to clinicians 210 having difficulty discerning relevant information from each of the alarms 220.

Additionally, while each of clinicians 210-a, 210-b, and 210-c may be responsible for monitoring each patient 105, in some cases, certain clinicians 210 may be responsible for only certain patients 105 or medical devices 110. Clinician 210-a may be a cardiologist and may be responsible for monitoring the heart rate of each patient 105 and may therefore not be concerned with alarms 220 (or medical devices 110) that are not related to heart rate of the patients 105. Clinician 210-b may only be responsible for patient 105-b (e.g., in intensive care) and may therefore not be concerned with alarms 220 associated with other patients 105-a, 105-c, and 105-d. Clinician 210-c may only be responsible for local patients, such as patient 105-d located in room 205-d in the same area as nurse station 120-a. Accordingly, as different clinicians 210 have different responsibilities, multiple sounding alarms 220 may not always be beneficial to each clinician 210 and could possibly cause clinicians 210 to naturally tune out multiple sounding alarms 220, not pay attention to most alarms 220, or have difficulty in determining whether an alarm 220 requires immediate attention by a certain clinician 210. The multiple sounding alarms 220 heard by multiple clinicians 210 who may not be responsible for responding to certain alarms 220 may lead to alarm fatigue for the clinicians 210.

In accordance with the present disclosure, a multi-layer alarm 220 may be generated having multiple audible layers of information related to the alarm 220, the patient 105, or the clinician responsible 210 for the patient 105. Each audible layer of the multi-layer alarm 220 may have corresponding layer properties which may include one or more of pitch, volume, and periodicity. Pitch may refer to the frequency of the acoustic wave associated with the layer when the multi-layer alarm is audibly transmitted. Volume may refer to the amplitude of the acoustic wave associated with the layer when the multi-layer alarm is audibly transmitted, and periodicity may refer to the number of times the acoustic wave is to be transmitted over a specific time interval.

The multi-layer alarm 220 may be generated by medical devices 110, nurse station 120-a, or computing device 115-a. A first audible layer of the multi-layer alarm may include information related to a measured parameter associated with a patient 105. For example, the first audible layer of the multi-layer alarm may identify the parameter of the patient 105 being measured by a medical device 110. For example, the first audible layer of the multi-layer alarm may indicate that the parameter of the patient 105 being monitored is a physiological parameter such as respiratory rate. The first audible layer may additionally, or alternatively, indicate the severity of the physiological parameter being measured based on the importance of the parameter (e.g., respiratory rate may be more severe than heart rate). In some cases, physiological parameters may be grouped into different severity categories based on how important the physiological parameter is to the health of the patient 105. For example, respiratory rate and heart rate may be grouped into a high severity category, oxygen saturation and temperature may be grouped into a medium severity category, and activity level may be grouped into a low severity category. In such instances, the first audible layer may indicate the severity of the physiological parameter and whether or not the physiological parameter is of high, medium, or low severity. The grouping of the physiological parameters may depend on the patient 105, the health of the patient 105, and the current condition or procedure in which the patient 105 is undergoing or has previously undergone. As such, different parameters may be grouped into different severity categories and other groupings of parameters may be considered without departing from the scope of the present disclosure.

In some embodiments, each of the physiological parameters within a severity group may have corresponding alarm thresholds of different criticalities (e.g., low criticality, medium criticality, and high criticality). For example, a medium severity physiological parameter, e.g., temperature, may be associated with a low criticality alarm threshold indicating that the temperature is slightly abnormal, but the patient 105 may not require immediate attention. The temperature may also be associated with a medium criticality alarm threshold indicating that the measured temperature is more abnormal (or worsening) and the patient 105 may soon require attention. Further, the temperature may also be associated with a high criticality alarm threshold and should the measured temperature reach the high criticality alarm threshold, the patient 105 may need immediate attention.

In some cases, a physiological parameter may be included in multiple severity categories based on the criticality of the alarm threshold corresponding the physiological parameter. For example, a physiological parameter such as heart rate may be associated with a low criticality alarm threshold, a medium criticality alarm threshold, and a high criticality alarm threshold. Heart rate along with a corresponding high criticality alarm threshold may be grouped into a high severity category, while the heart rate along with corresponding low and medium criticality alarm threshold may be grouped into a medium severity category. In another example, temperature may be associated with a high criticality alarm threshold and a low criticality alarm threshold and may be grouped based on the criticality of the alarm threshold. For instance, temperature along with a corresponding high criticality alarm threshold may be grouped into a high severity category, while temperature along with a corresponding low criticality alarm threshold may be grouped into a low severity category. Other groupings and corresponding criticalities of alarm thresholds associated with a measured physiological parameter may be considered without departing from the scope of the present disclosure.

A second audible layer of the multi-layer alarm may indicate other information associated with the medical device 110 or the patient 105, such as the clinician responsible for the patient 105, or the medical device 110 that triggered the alarm, or how long the patient 105 has been unattended. In some embodiments, the second audible layer of the multi-layer alarm may indicate that the measured physiological parameter has reached a particular alarm threshold (e.g., a low criticality alarm threshold, a medium criticality alarm threshold, or a high criticality alarm threshold). For example, the first audible layer of the multi-layer alarm may indicate severity of the measured physiological parameter, while the second audible layer of the multi-layer alarm may indicate the criticality of the measured physiological parameter. Thus, severity of the measured physiological parameter and the criticality of the measured physiological parameter may be indicated by the first and second audible layers of the multi-layer alarm.

In some instances, the severity and criticality of a physiological parameter may be indicated in a single audible layer. For example, a first audible layer may include layer properties (e.g., volume, pitch, periodicity) that indicates both the severity of the physiological parameter being measured and the criticality associated with the measured physiological parameter.

The multi-layer alarm may be sound at a medical device 110 associated with the alarm, at the nurse station 120-a, or at the computing device 115-a. The multi-layer alarm may then be repeated over a given time period. As the multi-layer alarm is generated with audible layers of information relating to the patient 105, the clinicians 210 may hear the multi-layer alarm and may be able to quickly discern relevant information (e.g., the severity of the alarm, the criticality of the alarm, which clinician 210 is responsible, whether the patient 105 requires immediate attention) related to the medical device 110, the parameter being measured, the patient, or the responsible clinician 210.

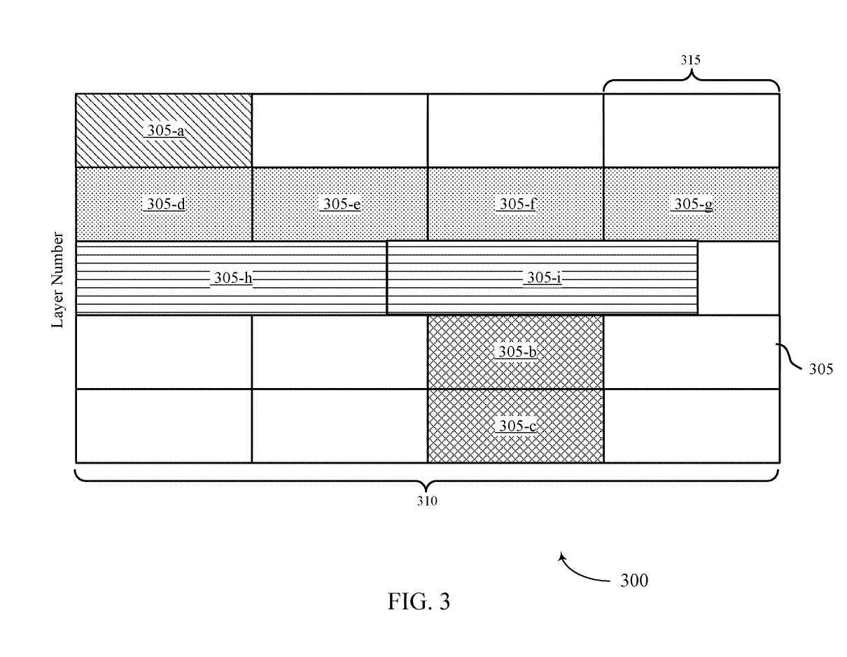

FIG. 3 illustrates an example of a multi-layer alarm 300 in accordance with aspects of the present disclosure. In some cases, the multi-layer alarm 300 may represent aspects of techniques performed by a medical device 110, computing device 115, or nurse station 120 as described with reference to FIGS. 1 and 2. For example, a medical device 110, a computing device 115, or a nurse station 120 may audibly transmit multi-layer alarm 300.

In FIG. 3, the multi-layer alarm 300 includes multiple audible layers 305 and spans a time period 310. Each of the multiple audible layers 305 span a corresponding time interval 315. Though the multi-layer alarm 300 spans time period 310, the multi-layer alarm 300 may be repeated when audibly transmitted over a given duration. For example, if time period 310 is ten seconds, the multi-layer alarm 300 may repeat six times over a one minute duration. Alternatively, the multi-layer alarm 300 may intermittently repeat over a duration (e.g., one time, two times, or three times over a one minute duration). The repetitions of multi-layer alarm 300 may be equally spaced over a duration or may be spaced differently over the duration. While a time period of ten seconds and a duration of one minute is described herein, any other time periods or durations may be considered without departing from the scope of the present disclosure.

Each layer 305 may indicate or identify information related to a patient such as a measured parameter, severity of the measured parameter, or a clinician responsible for the patient. In addition, each layer 305 may have corresponding layer properties such as pitch, volume, and periodicity to uniquely define or indicate such information and distinguish it from another layer 305. For example, layer 305-a may correspond with a given pitch and volume, but may not be repeated over the time period 310 (i.e., no periodicity). Layer 305-a may identify a measured parameter of the patient such as a physiological parameter or a severity of the measured parameter of the patient.

In one or more embodiments, multiple layers 305 may be used to indicate or identify information related to a patient. For example, layers 305-b and 305-c may each have corresponding layer properties that collectively may be used to indicate a measured parameter of the patient or a clinician responsible for the patient. In another example, layers 305-d, 305-e, 305-f, and 305-g may have the same pitch and volume properties and may repeat over time period 310 (i.e., periodic). In some cases, the time interval 315 over which a layer 305 spans may vary depending on the layer 305. For example, layers 305-h and 305-i span different time intervals compared to other layers 305 of the multi-layer alarm 300 and may repeat twice over time period 310.

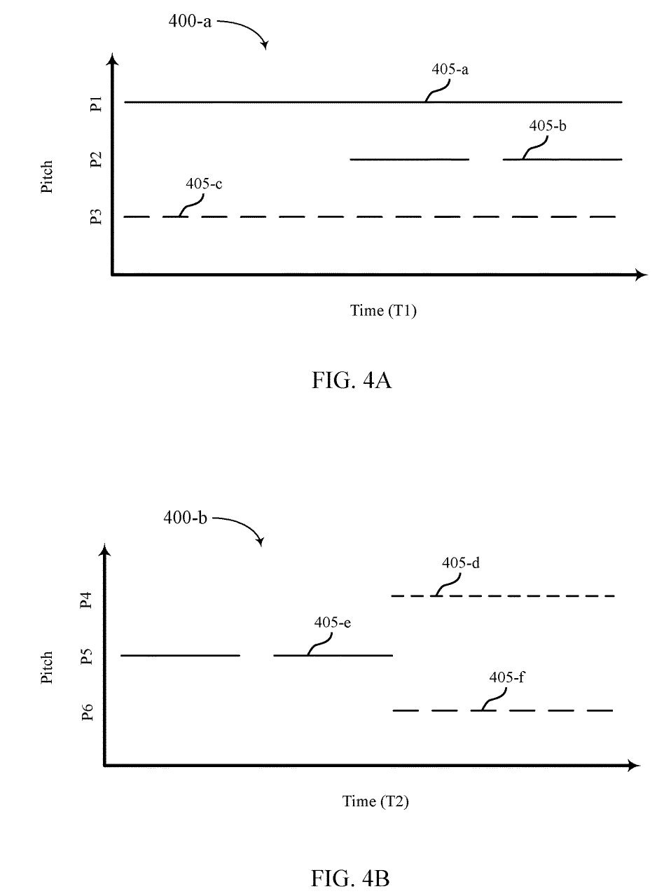

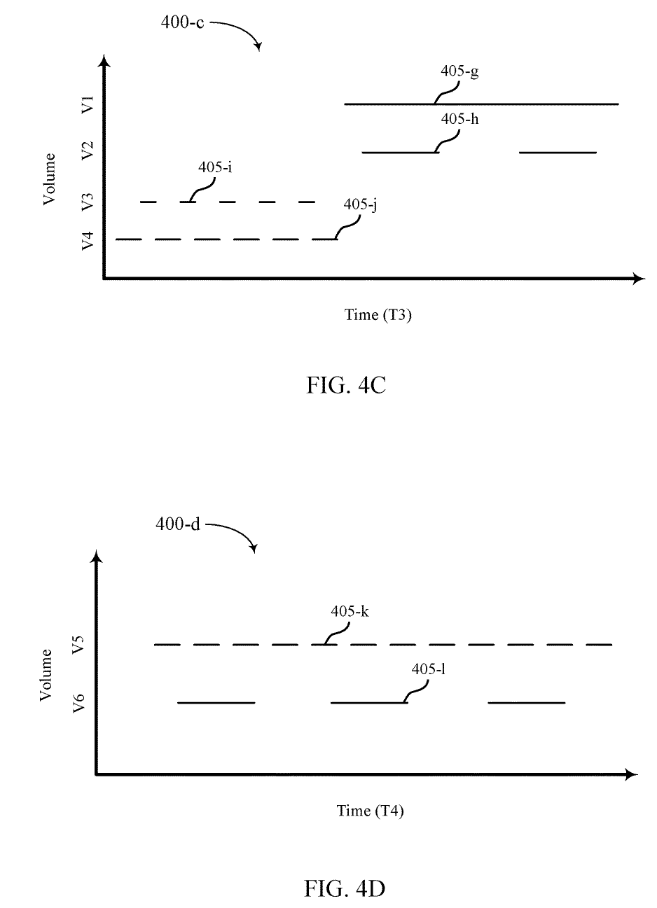

FIGS. 4A-4D illustrate examples of multi-layer alarms 400 in accordance with aspects of the present disclosure. In some cases, the multi-layer alarms 400 may represent aspects of techniques performed by a medical device 110, computing device 115, or nurse station 120 as described with reference to FIGS. 1 and 2. For example, a medical device 110, a computing device 115, or a nurse station 120 may audibly transmit multi-layer alarms 400.

In FIG. 4A, a multi-layer alarm 400-a is shown spanning time period T1 and includes layers 405-a, 405-b, and 405-c. Each of the layers 405-a, 405-b, and 405-c may indicate information associated with a patient being monitored by a medical device (e.g., medical device 110) and include corresponding layer properties such as pitch and periodicity. As shown, layer 405-a has a pitch P1 and is continuous over time period T1. Layer 405-b has a pitch P2 and repeats twice over time period T1. However, layer 405-b begins after a delay. Layer 405-c has a pitch P3 and repeats thirteen times over time period T1. In some embodiments, two or more of layers 405-a, 405-b, and 405-c may collectively indicate information associated with the patient. For example, layer 405-a may indicate the responsible clinician for a particular patient, layer 405-b may indicate the physiological parameter that triggered the alarm, and layer 405-c may indicate where the patient is located within the healthcare facility.

In FIG. 4B, a multi-layer alarm 400-b is shown spanning time period T2 and includes layers 405-d, 405-e, and 405-f. Each of the layers 405-d, 405-e, and 405-f may indicate information associated with a patient being monitored by a medical device (e.g., medical device 110) and include corresponding layer properties such as pitch and periodicity. As shown, layer 405-d has a pitch P4 and repeats twelve times over time period T2, but begins after a delay. Layer 405-e has a pitch P5 and repeats twice over time period T2, but ends with a delay. Layer 405-f has a pitch P6 and repeats six times over time period T2 and also begins after a delay. In some embodiments, two or more of layers 405-d, 405-e, and 405-f may collectively indicate information associated with the patient. For example, layer 405-d may indicate the physiological parameter that triggered the alarm, layer 405-e may indicate that the measured physiological parameter has escalated recently (e.g., the measured parameter or overall condition of the patient was worsened), and layer 405-f may indicate the clinician responsible for the patient.

In FIG. 4C, a multi-layer alarm 400-c is shown spanning time period T3 and includes layers 405-g, 405-h, 405-i and 405-j. Each of the layers 405-g, 405-h, 405-i and 405-j may indicate information associated with a patient being monitored by a medical device (e.g., medical device 110) and include corresponding layer properties such as volume and periodicity. As shown, layer 405-g has a volume V1 and is continuous after a delay over time period T3. Layer 405-h has a Volume V2 and repeats twice over time period T3, but begins with a delay. Layer 405-i has a volume V3 and repeats five times over time period T3 but ends with a delay. Layer 405-j has a volume V4 and repeats six times over time period T3 but ends with a delay. In some embodiments, two or more of layers 405-g, 405-h, 405-i and 405-j may collectively indicate information associated with the patient. For example, layer 405-g may indicate the severity (e.g., high, medium, or low severity) associated with the alarm, layer 405-h may indicate the clinician responsible for the patient, layer 405-i may indicate that the patient has been left unattended for a certain amount of time, and layer 405-j may indicate the device type that triggered the alarm.

In FIG. 4D, a multi-layer alarm 400-d is shown spanning time period T4 and includes layers 405-k and 405-1. Each of the layers 405-k and 405-1 may indicate information associated with a patient being monitored by a medical device (e.g., medical device 110) and include corresponding layer properties such as volume and periodicity. As shown, layer 405-k has a volume V5 and repeats twelve times over time period T4, but begins with a delay. Layer 405-1 has a volume V6 and repeats three times over time period T4 but begins and ends with a delay. In some embodiments, layers 405-k and 405-1 may collectively indicate information associated with the patient. For example, layer 405-k may indicate the severity (e.g., high, medium, or low severity) or the criticality associated with a physiological parameter that triggered the alarm, and layer 405-1 may indicate location of the patient.

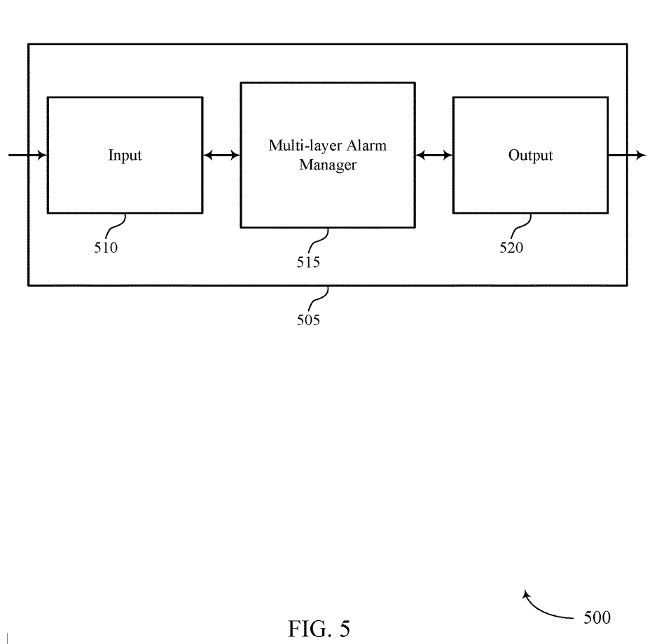

FIG. 5 shows a block diagram 500 of a device 505 that supports multi-layer alarming in accordance with various aspects of the present disclosure. Device 505 may be an example of aspects of a medical device 110, computing device 115, or nurse station 120, as described with reference to FIGS. 1 and 2.

As shown, device 505 may include input 510, multi-layer alarm manager 515, and output 520. Device 505 may also include a processor. Each of these components may be in communication with one another (e.g., via one or more buses).

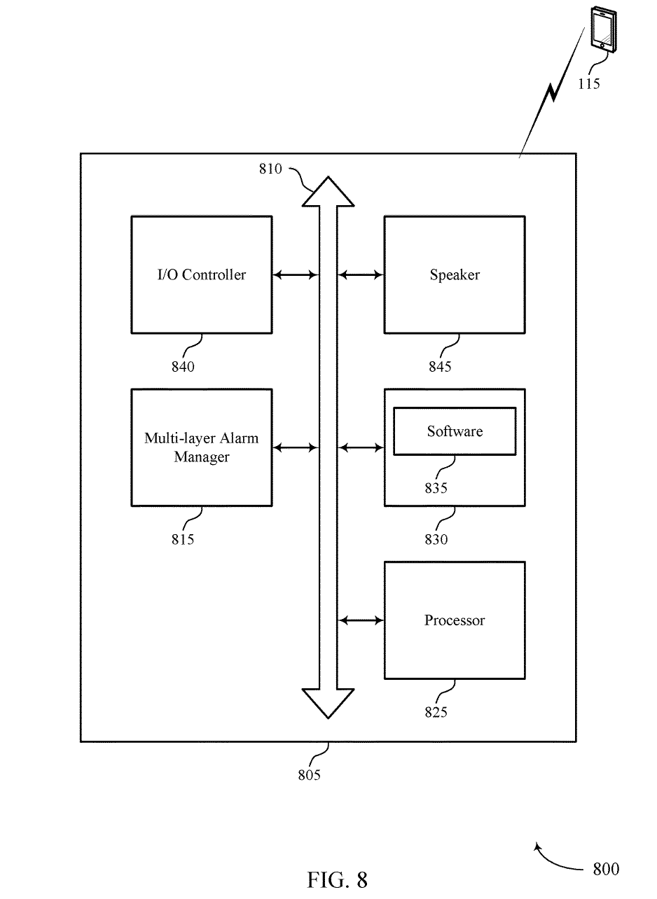

Multi-layer alarm manager 515 may encode a multi-layer alarm associated with a patient. The multi-layer alarm manager 515 may encode a first audible layer of the multi-layer alarm, the first audible layer identifying a severity of a measured parameter of the patient and encode a second audible layer of the multi-layer alarm, the second audible layer indicating information other than the identified severity of the measured parameter. The multi-layer alarm manager 515 may also audibly transmit the multi-layer alarm. In some embodiments, multi-layer alarm manager 515 may be an example of aspects of the multi-layer alarm manager 815 described with reference to FIG. 8.

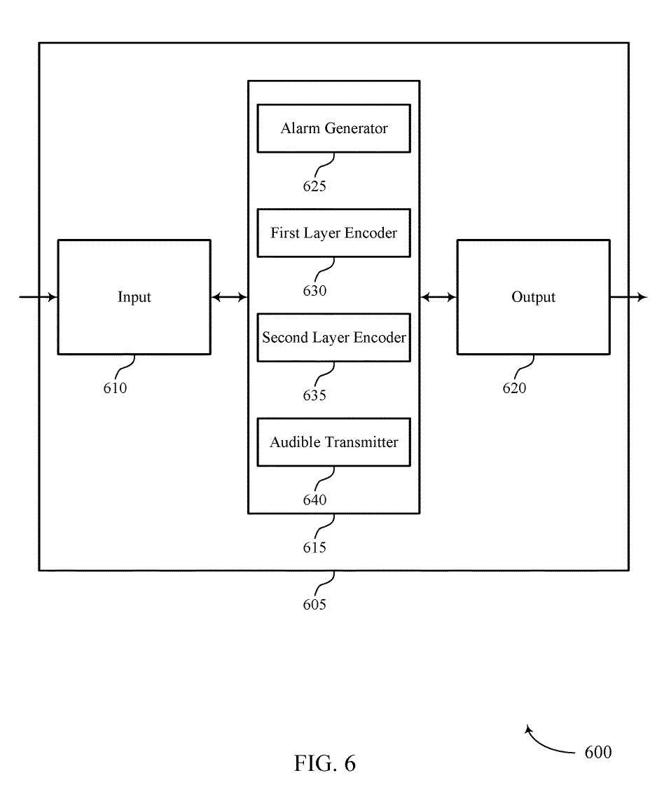

FIG. 6 shows a block diagram 600 of a device 605 that supports multi-layer alarming in accordance with various aspects of the present disclosure. Device 605 may be an example of aspects of a device 505 or a medical device 110 as described with reference to FIGS. 1, 2 and 5. Device 605 may include input 610, multi-layer alarm manager 615, and output 620. Device 605 may also include a processor. Each of these components may be in communication with one another (e.g., via one or more buses).

Multi-layer alarm manager 615 may include alarm generator 625, first layer encoder 630, second layer encoder 635, and audible transmitter 640. Alarm generator 625 may encode a multi-layer alarm associated with a patient. In one or more embodiments, encoding the multi-layer alarm is based on a measured parameter (e.g., measured by a medical device 110) crossing a threshold. In some cases, encoding the multi-layer alarm may be based on a status or a technical issue associated with device 605 or other device (e.g., medical device 110, nurse station 120, computing device 115). In some cases, encoding the multi-layer alarm includes determining one or more layer properties for each audible layer of the multi-layer alarm such as a volume, a pitch, or a periodicity.

First layer encoder 630 may encode a first audible layer of the multi-layer alarm, the first audible layer identifying a severity of a measured parameter of the patient. The first layer audible layer may be uniquely defined by one or more layer properties to identify the severity of the measure parameter of the patient.

Second layer encoder 635 may encode a second audible layer of the multi-layer alarm, the second audible layer indicating information other than the identified severity of the measured parameter. In some cases, the information other than the identified severity of the measured parameter includes a clinician responsible for the patient or a duration of alarm transmission without clinician intervention. In one or more embodiments, the second audible layer may include an escalation level associated with the measured parameter, a device type associated with measuring the measured parameter, or a technical issue of a medical device (e.g., medical device 110) associated with the patient. The second audible layer may also indicate a patient location or a criticality of the measured physiological parameter.

Audible transmitter 640 may audibly transmit the multi-layer alarm. In some cases, audibly transmitting the multi-layer alarm may include audibly transmitting both the first and second audible layers contemporaneously or sequentially or periodically over a given time interval.

In one or more embodiments, multi-layer alarm manager 615 may be an example of aspects of the multi-layer alarm manager 815 described with reference to FIG. 8.

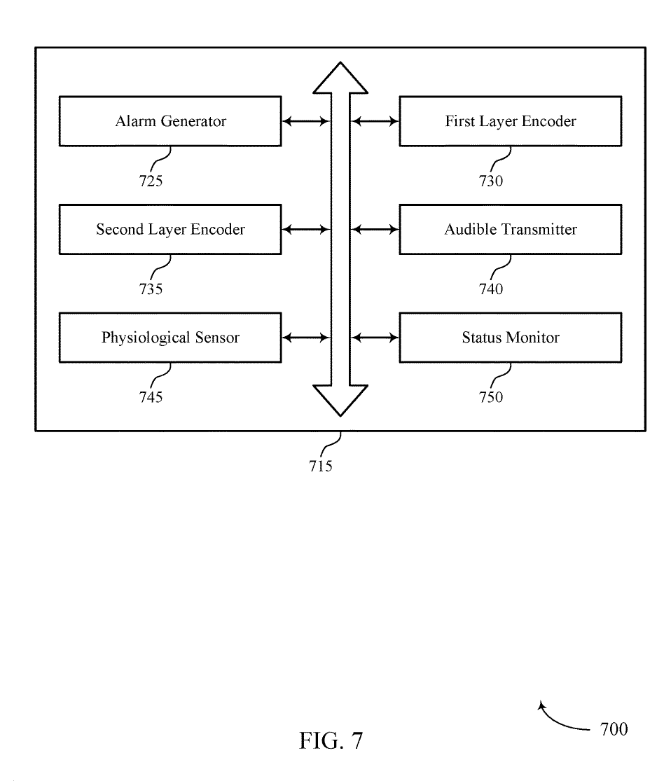

FIG. 7 shows a block diagram 700 of a multi-layer alarm manager 715 that supports multi-layer alarming in accordance with various aspects of the present disclosure. The multi-layer alarm manager 715 may be an example of aspects of a multi-layer alarm manager 515, a multi-layer alarm manager 615, or a multi-layer alarm manager 815 described with reference to FIGS. 5, 6, and 8. The multi-layer alarm manager 715 may include alarm generator 725, first layer encoder 730, second layer encoder 735, and audible transmitter 740. Each of these modules may communicate, directly or indirectly, with one another (e.g., via one or more buses).

Alarm generator 725 may encode a multi-layer alarm associated with a patient. In one or more embodiments, encoding the multi-layer alarm is based on a measured parameter (e.g., measured by a medical device 110) crossing a threshold. In some cases, encoding the multi-layer alarm may be based on a status or a technical issue associated with a device (e.g., medical device 110, nurse station 120, computing device 115). In some cases, encoding the multi-layer alarm includes determining one or more layer properties for each audible layer of the multi-layer alarm such as a volume, a pitch, or a periodicity.

First layer encoder 730 may encode a first audible layer of the multi-layer alarm, the first audible layer identifying a severity of a measured parameter of the patient. The first layer audible layer may be uniquely defined by one or more layer properties to identify the severity of the measured parameter of the patient.

Second layer encoder 735 encode a second audible layer of the multi-layer alarm, the second audible layer indicating information other than the identified severity of the measured parameter. In some cases, the information other than the identified severity of the measured parameter includes a clinician responsible for the patient or a duration of alarm transmission without clinician intervention. In one or more embodiments, the second audible layer may include an escalation level associated with the measured parameter, a device type associated with measuring the measured parameter, or a technical issue of a medical device (e.g., medical device 110) associated with the patient. The second audible layer may also indicate a patient location or a criticality of the measured parameter.

Audible transmitter 740 may audibly transmit the multi-layer alarm. In some cases, audibly transmitting the multi-layer alarm may include audibly transmitting both the first and second audible layers contemporaneously or sequentially or periodically over a given time interval.

Physiological sensor 745 may measure a parameter of the patient (e.g., using medical device 110). In some cases, the measured parameter includes a physiological parameter of the patient such as a heart rate, a respiratory rate, a blood pressure, a temperature, an activity level, or an oxygen saturation level.

Status monitor 750 may determine a status of one or more devices (e.g., medical device 110, nurse station 120, or computing device 115). For example, the status monitor 750 may determine a low battery indication associated with the device or a loss of power to the device. The status monitor 750 may also determine a self-test failure associated with the device, a startup failure of the device, or other technical issue associated with the device.

FIG. 8 shows a diagram of a system 800 including a device 805 that supports multi-layer alarming in accordance with various aspects of the present disclosure. Device 805 may be an example of a device 505, device 605, a medical device 110, nurse station 120, or computing device 115, as described above, e.g., with reference to FIGS. 1, 2, 5, and 6.

Device 805 may include components for bi-directional voice and data communications or components for transmitting and receiving communications. Device 805 may also include multi-layer alarm manager 815, processor 825, memory 830, software 835, I/O controller 840, and speaker 845.

Processor 825 may include an intelligent hardware device, (e.g., a central processing unit (CPU), a microcontroller, an application specific integrated circuit (ASIC), etc.)

Memory 830 may include random access memory (RAM) and read only memory (ROM). The memory 830 may store computer-readable, computer-executable software 835 including instructions that, when executed, cause the processor to perform various functions described herein. In some cases, the memory 830 may contain, among other things, a Basic Input-Output system (BIOS) which may control basic hardware and/or software operation such as the interaction with peripheral components or devices.

Software 835 may include code to implement aspects of the present disclosure, including code to support multi-layer alarming. Software 835 may be stored in a non-transitory computer-readable medium such as system memory or other memory. In some cases, the software 835 may not be directly executable by the processor but may cause a computer (e.g., when compiled and executed) to perform functions described herein.

I/O controller 840 may manage input and output signals for device 805. In some cases, I/O controller 840 may utilize an operating system such as iOS®, ANDROID®, MS-DOS®, MS-WINDOWS®, OS/2®, UNIX®, LINUX®, or another known operating system.

Speaker 845 may audibly transmit an alarm from device 805. In some cases, the speaker 845 may audibly transmit a multi-layer alarm having multiple layers, each associated with different layer properties, as described above with reference to FIGS. 3 and 4A-4D.

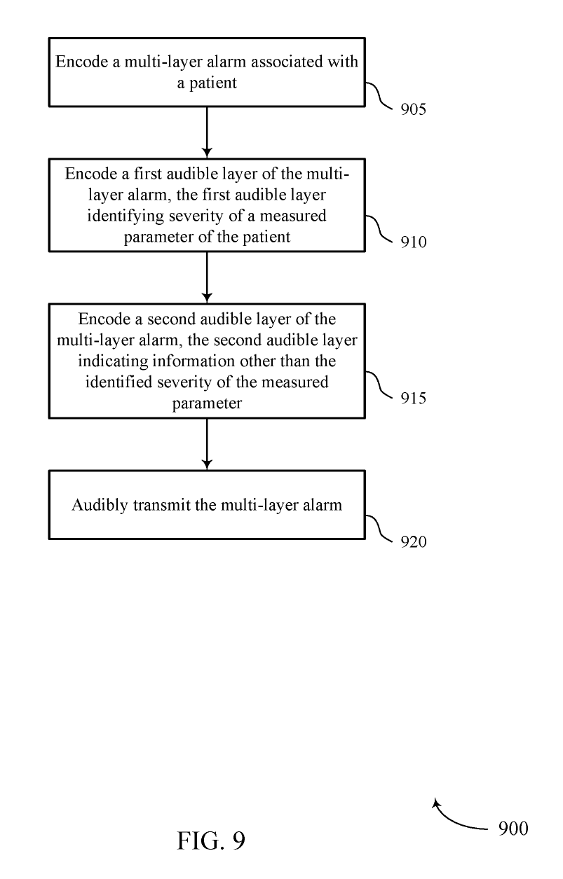

FIG. 9 shows a flowchart illustrating a method 900 for multi-layer alarming in accordance with various aspects of the present disclosure. The operations of method 900 may be implemented by a device (e.g., medical device 110, nurse station 120, computing device 115), or its components as described herein. For example, the operations of method 900 may be performed by a multi-layer alarm manager as described with reference to FIGS. 5 through 7. In some examples, a device may execute a set of codes to control the functional elements of the device to perform the functions described below. Additionally or alternatively, the device may perform aspects the functions described below using special-purpose hardware.

At block 905, the device may encode a multi-layer alarm associated with a patient. The operations of block 905 may be performed according to the methods described with reference to FIGS. 2 through 4. In certain examples, aspects of the operations of block 905 may be performed by an alarm generator as described with reference to FIGS. 5 through 7.

At block 910, the device may encode a first audible layer of the multi-layer alarm, the first audible layer identifying a severity of a measured parameter of the patient. The operations of block 910 may be performed according to the methods described with reference to FIGS. 2 through 4. In certain examples, aspects of the operations of block 910 may be performed by a first layer encoder as described with reference to FIGS. 5 through 7.

At block 915, the device may encode a second audible layer of the multi-layer alarm, the second audible layer indicating information other than the identified severity of the measured parameter. The operations of block 915 may be performed according to the methods described with reference to FIGS. 2 through 4. In certain examples, aspects of the operations of block 915 may be performed by a second layer encoder as described with reference to FIGS. 5 through 7.

At block 920, the device may audibly transmit the multi-layer alarm. The operations of block 920 may be performed according to the methods described with reference to FIGS. 2 through 4. In certain examples, aspects of the operations of block 920 may be performed by an audible transmitter as described with reference to FIGS. 5 through 7.

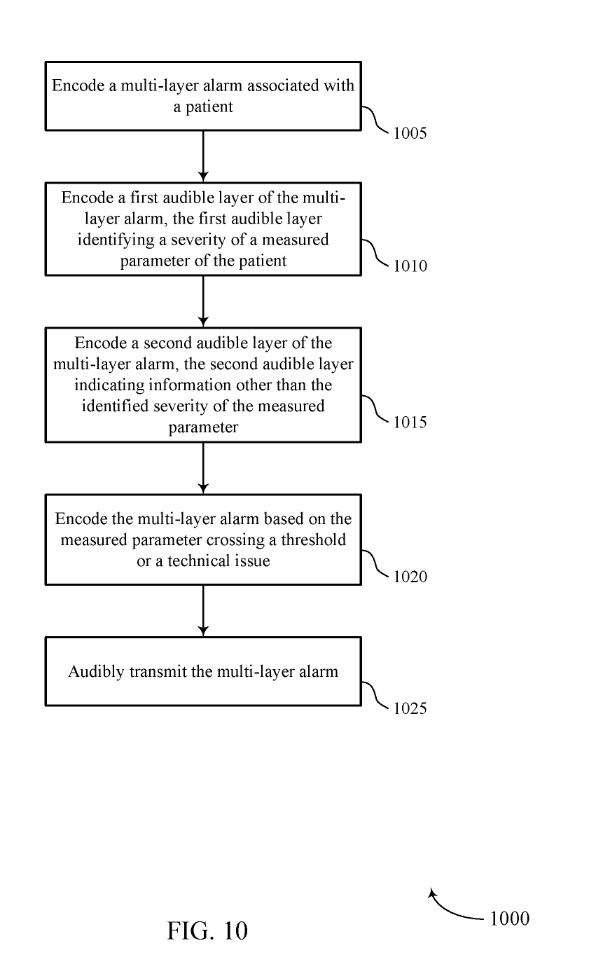

FIG. 10 shows a flowchart illustrating a method 1000 for multi-layer alarming in accordance with various aspects of the present disclosure. The operations of method 1000 may be implemented by a device (e.g., medical device 110, nurse station 120, or computing device 115) or its components as described herein. For example, the operations of method 1000 may be performed by a multi-layer alarm manager as described with reference to FIGS. 5 through 7. In some examples, a device may execute a set of codes to control the functional elements of the device to perform the functions described below. Additionally or alternatively, the device may perform aspects the functions described below using special-purpose hardware.

At block 1005, the device may encode a multi-layer alarm associated with a patient. The operations of block 1005 may be performed according to the methods described with reference to FIGS. 2 through 4. In certain examples, aspects of the operations of block 1005 may be performed by an alarm generator as described with reference to FIGS. 5 through 7.

At block 1010, the device may encode a first audible layer of the multi-layer alarm, the first audible layer identifying a severity of a measured parameter of the patient. The operations of block 1010 may be performed according to the methods described with reference to FIGS. 2 through 4. In certain examples, aspects of the operations of block 1010 may be performed by a first layer encoder as described with reference to FIGS. 5 through 7.

At block 1015, the device may encode a second audible layer of the multi-layer alarm, the second audible layer indicating information other than the identified severity of the measured parameter. The operations of block 1015 may be performed according to the methods described with reference to FIGS. 2 through 4. In certain examples, aspects of the operations of block 1015 may be performed by a second layer encoder as described with reference to FIGS. 5 through 7.

At block 1020, the device may encode the multi-layer alarm is based on the measured parameter crossing a threshold or a technical issue. The operations of block 1020 may be performed according to the methods described with reference to FIGS. 2 through 4. In certain examples, aspects of the operations of block 1020 may be performed by an alarm generator as described with reference to FIGS. 5 through 7.

At block 1025, the device may audibly transmit the multi-layer alarm. The operations of block 1025 may be performed according to the methods described with reference to FIGS. 2 through 4. In certain examples, aspects of the operations of block 1025 may be performed by an audible transmitter as described with reference to FIGS. 5 through 7.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C).

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable read only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1-14. (canceled)

15. The system of claim 25, wherein the measured physiological parameter of the patient comprises a heart rate, a respiratory rate, a blood pressure, a temperature, an activity level, or an oxygen saturation level.

16. The system of claim 25, wherein the multi-layer alarm manager is further configured to:
encode the multi-layer alarm data structure based at least in part on a measured physiological parameter crossing one or more thresholds or technical issues of one or more medical devices;
encode a single data layer with one or more layer properties that correspond to one or more thresholds and groupings for a measured physiological parameter; and
encode a single data layer with one or more layer properties that correspond to patient information and technical issues from one or more medical devices.

17. The system of claim 16, wherein the technical issues comprise a low battery indication associated with the medical device, a loss of power to the medical device, a self-test failure associated with the medical device, or a startup failure of the medical device.

18. The system of claim 25, wherein the multi-layer alarm manager is further configured to:
generate one or more layer properties for each of the at least two data layers of the multi-layer alarm data structure, wherein one or more layer properties define and differentiate each data layer from another data layer of the multi-layer alarm.

19-21. (canceled)

22. The system of claim 25, wherein the encoded information of the at least two data layers comprises a clinician responsible for the patient, a duration of alarm transmission without clinician intervention, an escalation level associated with the measured physiological parameter, a device type associated with measuring the measured physiological parameter, a technical issue of a medical device associated with the patient, a criticality of the measured physiological parameter, or a patient location.

23. (canceled)

24. The system of claim 25, wherein audibly transmitting the multi-layer alarm data structure comprises:
audibly transmitting the first data layer after audibly transmitting subsequent data layers.

25. A system for alerting a clinician, comprising:
a multi-layer alarm manager configured to generate a transmittable multi-layer alarm data structure, the multi-layer alarm data structure comprising at least two data layers of encoded information related to a patient, each of the at least two data layers comprising one or more layer properties, the one or more layer properties comprising pitch, volume and periodicity, the first layer of the at least two data layers corresponding to a patient's measured physiological parameter, the second layer of the at least two data layers corresponding to a medical device, wherein the at least two data layers are configured to be sounded by an alarm generator simultaneously to generate an audibly transmitted alarm.

26. The system of claim 16, wherein the groupings comprise:
criticality and severity of a measured physiological parameter, based on the importance of the particular physiological parameter to a specific patient.
